(19) United States
(12) Patent Application Publication
(10) Pub. No.: US 2025/0352681 A1
(43) Pub. Date: Nov. 20, 2025

(54) Glove Sanitizer

(71) Applicant: Barry Murphy, Athy (IE)

(72) Inventor: Barry Murphy, Athy (IE)

(21) Appl. No.: 18/734,548

(22) Filed: Jun. 5, 2024

Related U.S. Application Data
(60) Provisional application No. 63/577,982, filed on Jun. 12, 2023.

(51) Int. Cl. A61L 2/08; A47G 25/90; A61L 2/18; A61L 2/24

(52) U.S. Cl. CPC A61L 2/08; A47G 25/904; A61L 2/18; A61L 2/24; A61L 2202/11; A61L 2202/122; A61L 2202/17; A61L 2202/26

(57) ABSTRACT

An aspect of some embodiments of the present invention relates to a glove sanitizer, comprising a case, a glove removal apparatus, a ventilation system, and a lighting system. The case has a wall with at least one opening configured for insertion of a gloved hand therethrough. The glove removal apparatus includes a curved pipe and a pump. The curved pipe has suction holes positioned along the pipe, each suction hole being positioned to contact a respective fingertip of the glove when the gloved hand is inserted into the case. The pump is configured to suck air from the curved pipe, thereby suctioning the tips of the glove at the suction holes. The ventilation system directs an air flow at the glove, to dry an outer surface of the glove. The lighting system to emits light inside the case to disinfect an outside of the glove inside the case.

### CROSS-REFERENCES TO RELATED APPLICATIONS

This application claims priority from U.S. Provisional Application Ser. No. 63/577,982 filed Jun. 6, 2024, which is hereby incorporated herein by reference in the respective in its entirety.

### TECHNICAL FIELD

This invention relates to a glove sanitizer, for removing gloves from a user and sanitizing the gloves.

### BACKGROUND OF THE INVENTION

Wearing gloves for hygiene purposes and to prevent infection has become standard practice in many industries. In particular, in the transport industry, drivers of vehicles such as heavy goods vehicles and public transport vehicles must wear work gloves to protect themselves and others from infection from germs or microbes such as bacteria, viruses and the like. Work gloves can quickly become contaminated, both inside and outside, and must be changed at regular intervals.

However, frequent changing of gloves can give rise to significant costs while new replacement gloves are not always available to drivers. In addition, it is not possible for drivers to effectively dry and thoroughly disinfect soiled gloves manually and with confidence. Even if work gloves are biodegradable for environmental reasons, difficulties in effectively cleaning the gloves can result in repeated replacement of the gloves and increased use of raw materials.

An object of the invention is to provide a sanitizer that can be used to sanitize work gloves such as biodegradable and work gloves.

### BRIEF SUMMARY OF EMBODIMENTS OF THE INVENTION

An aspect of some embodiments of the present invention relates to a glove sanitizer, comprising a case, a glove removal apparatus, a ventilation system, and a lighting system. The case has a wall with at least one opening configured for insertion of a gloved hand therethrough. The glove removal apparatus is designed for removing the glove from the gloved hand and retain the glove inside the case when the hand is moved out of the case. The glove removal apparatus include a curved pipe and a pump. The curved pipe has suction holes positioned along the pipe, each suction hole being positioned to contact a respective fingertip of the glove when the gloved hand is inserted into the case. The pump is configured to suck air from the curved pipe, thereby suctioning the tips of the glove at the suction holes. The ventilation system is configured to direct an air flow at the glove retained inside the case, to dry an outer surface of the glove. The lighting system is configured to emit light inside the case having a predetermined wavelength or combination of wavelengths to disinfect an outside of the glove inside the case.

In a variant, the glove sanitizer further includes at least one tab extending inside the case near the at least one opening, the tab being configured to enter a space inside a cuff of the glove and having an outlet configured to blow air into the glove to separate the glove from the hand and to dry an inside of the glove.

In another variant, the tab further comprises a lamp configured to emit light inside the case having a predetermined wavelength or combination of wavelengths to disinfect the inside of the glove.

In yet another variant, the at least one tab has an upper surface facing a ceiling of the case and a lower surface facing a floor of the case, wherein the outlet is located on the upper surface of the tab, such that a direction of the air blown by the outlet is at least partially upwards in a direction of the ceiling.

In a further variant, the glove sanitizer further comprises: a disinfectant tank configured to containing a disinfectant liquid; and a disinfectant delivery system configured to direct a spray of the disinfectant liquid onto the glove inside the case.

In yet a further variant, the case has a transparent ceiling, to enable a user to see through the ceiling and move the user's gloved hand inside the case to position the fingertips of the glove to contact the respective suction holes.

In a variant, the glove sanitizer further comprises a sensor for sensing entry of the gloved hand into the case and, responsive to the sensing, activating the pump.

In yet another variant, the glove sanitizer further comprises a control unit configured to: responsive to a sensing of an entry of the gloved hand by the sensor, activate the pump and the outlet assembly to remove the glove from the user's hand; responsive to a user input, disinfect and dry the glove retained inside the case, by: activating the lighting system; activating the ventilation system.

In a further variant, the glove sanitizer further comprises: a disinfectant tank configured to containing a disinfectant liquid; and a disinfectant delivery system configured to direct a spray of the disinfectant liquid onto the glove inside the case. Responsive to a sensing of an entry of the gloved hand by the sensor, the control unit is configured to activate the pump and the outlet assembly to remove the glove from the user's hand. Responsive to a user input, the control unit is configured to disinfect and dry the glove retained inside the case, by: activating the disinfectant delivery system; activating the lighting system; activating the ventilation system.

In a variant, the glove sanitizer further comprises at least one tab extending inside the case near the at least one opening, the tab being configured to enter a space inside a cuff of the glove and having an outlet configured to blow air into the glove to separate the glove from the hand and to dry an inside of the glove, the tab further comprises a lamp configured to emit light inside the case having a predetermined wavelength or combination of wavelengths to disinfect the inside of the glove. Responsive to the user input, the control unit is configured to disinfect and dry the glove retained inside the case, by: activating the lighting system and the lamp of the tab; activating the ventilation system.

In yet another variant, the glove sanitizer further comprises: a disinfectant tank configured to containing a disinfectant liquid; and a disinfectant delivery system configured to direct a spray of the disinfectant liquid onto the glove inside the case. Responsive to a sensing of an entry of the gloved hand by the sensor, the control unit is configured to activate the pump and the outlet assembly to remove the glove from the user's hand. Responsive to a user input, the control unit is configured to disinfect and dry the glove retained inside the case, by: activating the disinfectant delivery system; activating the lighting system and the lamp of the tab; activating the ventilation system.

Other features and aspects of the invention will become apparent from the following detailed description, taken in conjunction with the accompanying drawings, which illustrate, by way of example, the features in accordance with embodiments of the invention. The summary is not intended to limit the scope of the invention, which is defined solely by the claims attached hereto.

### BRIEF DESCRIPTION OF THE DRAWINGS

The present invention, in accordance with one or more various embodiments, is described in detail with reference to the following figures. The drawings are provided for purposes of illustration only and merely depict typical or example embodiments of the invention. These drawings are provided to facilitate the reader's understanding of the invention and shall not be considered limiting of the breadth, scope, or applicability of the invention. It should be noted that for clarity and ease of illustration these drawings are not necessarily made to scale.

Some of the figures included herein illustrate various embodiments of the invention from different viewing angles. Although the accompanying descriptive text may refer to such views as “top,” “bottom” or “side” views, such references are merely descriptive and do not imply or require that the invention be implemented or used in a particular spatial orientation unless explicitly stated otherwise.

FIG. 1 is a perspective view of a glove sanitizer, according to some embodiments of the present invention;

FIGS. 2-7 are schematic views of the inside of the glove sanitizer, illustrating the process by which a glove is removed from a user's hand and retained in the box for sanitizing, according to some embodiments of the present invention;

FIGS. 8 and 9 schematic drawings showing the inside of the glove sanitizer from above, according to some embodiments of the present invention;

FIG. 10 is a perspective drawing of a glove sanitizer which includes a disinfectant tank, according to some embodiments of the present invention;

FIG. 11 is an isometric view of a cutout of the glove sanitizer, according to some embodiments of the present invention.

FIG. 12 is a block diagram showing a control unit controlling different elements of the glove sanitizer according to inputs, according to some embodiments of the present invention; and

FIG. 13 illustrates an example of a tab tilted downward, according sot some embodiments of the present invention.

The figures are not intended to be exhaustive or to limit the invention to the precise form disclosed. It should be understood that the invention can be practiced with modification and alteration, and that the invention be limited only by the claims and the equivalents thereof.

### DETAILED DESCRIPTION OF THE EMBODIMENTS OF THE INVENTION

From time-to-time, the present invention is described herein in terms of example environments. Description in terms of these environments is provided to allow the various features and embodiments of the invention to be portrayed in the context of an exemplary application. After reading this description, it will become apparent to one of ordinary skill in the art how the invention can be implemented in different and alternative environments.

Unless defined otherwise, all technical and scientific terms used herein have the same meaning as is commonly understood by one of ordinary skill in the art to which this invention belongs. All patents, applications, published applications and other publications referred to herein are incorporated by reference in their entirety. If a definition set forth in this section is contrary to or otherwise inconsistent with a definition set forth in applications, published applications and other publications that are herein incorporated by reference, the definition set forth in this document prevails over the definition that is incorporated herein by reference.

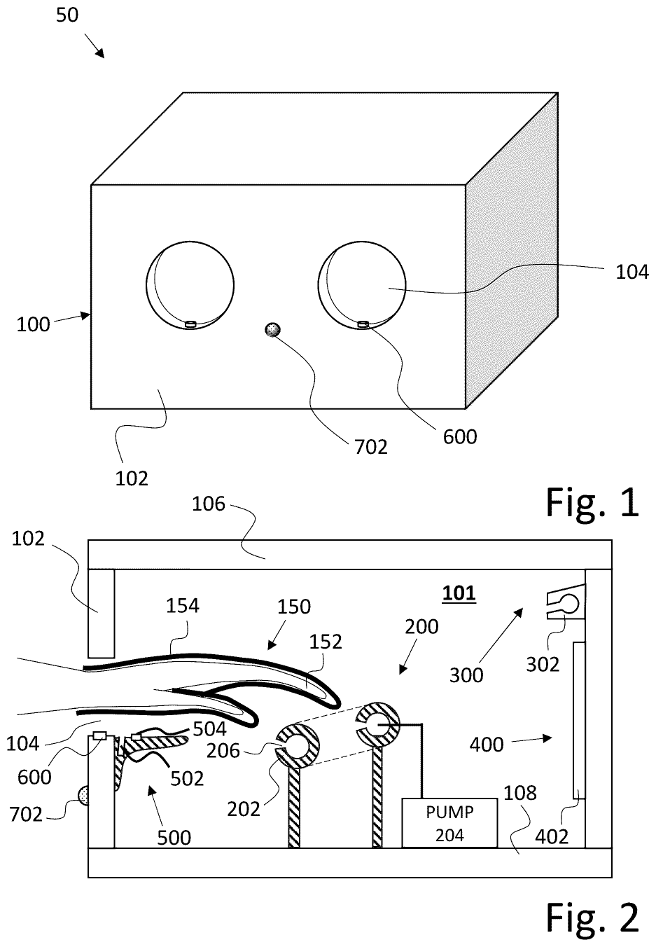

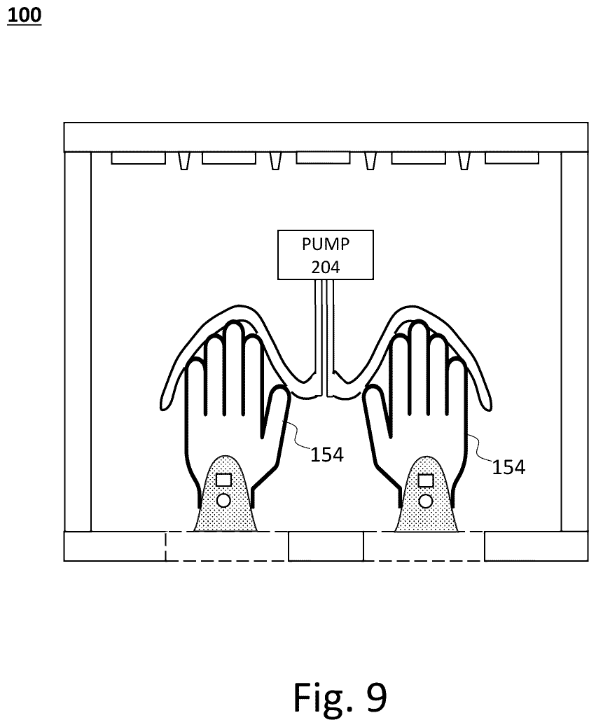

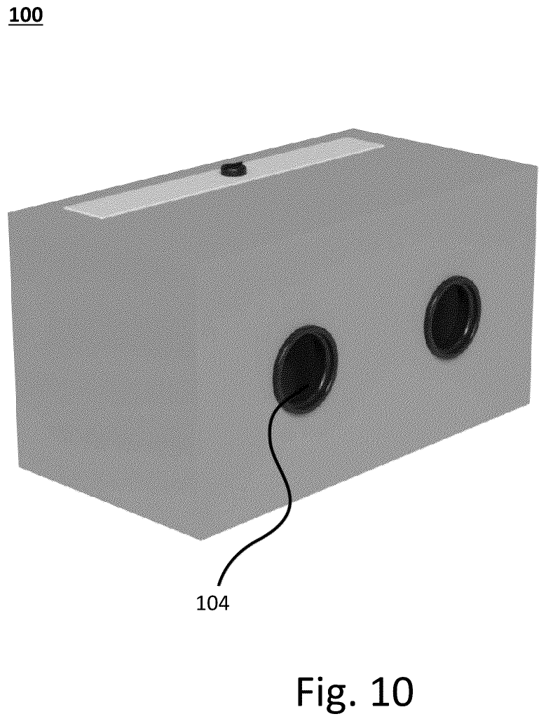

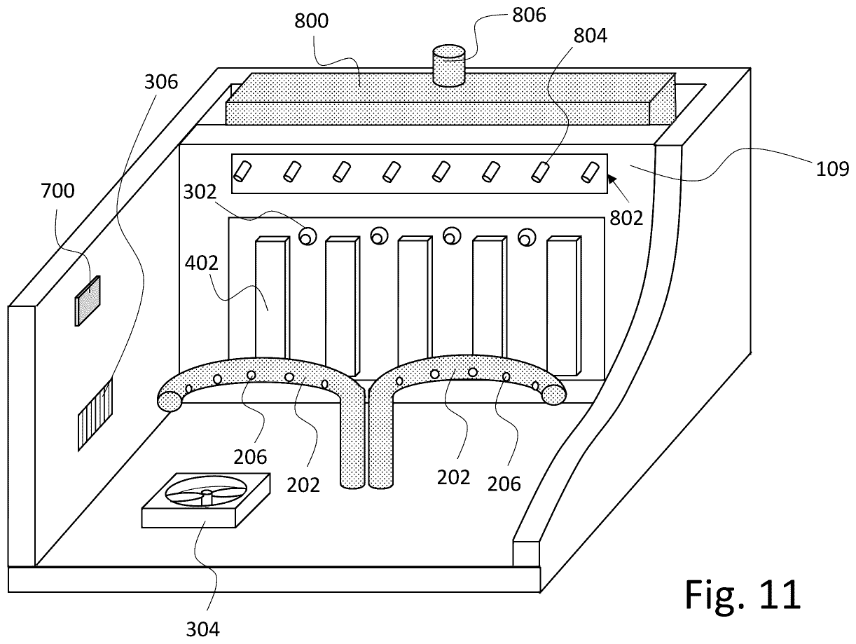

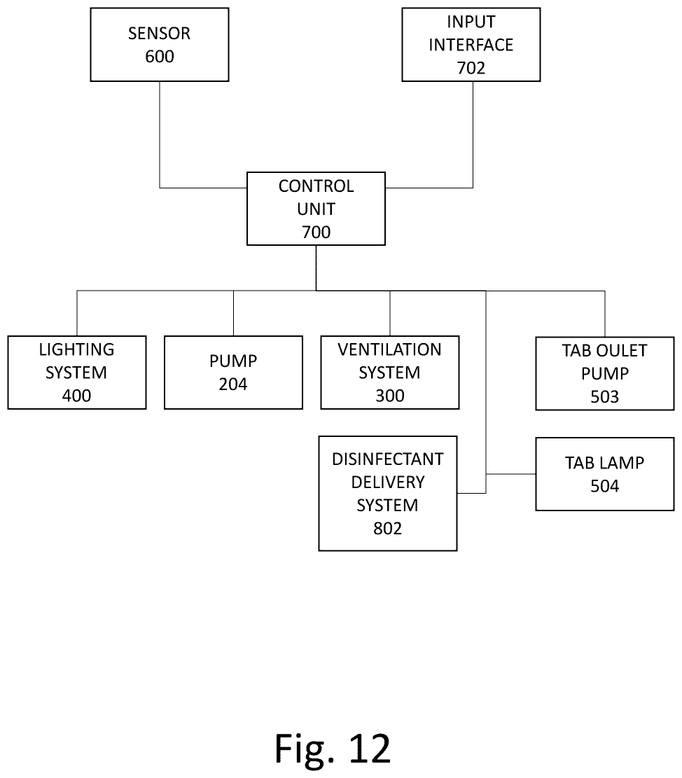

Referring now to the drawings, FIG. 1 is a perspective view of a glove sanitizer 100, according to some embodiments of the present invention. FIGS. 2-7 are schematic views of the inside of the glove sanitizer, illustrating the process by which a glove is removed from a user's hand and retained in the box for sanitizing, according to some embodiments of the present invention. FIGS. 8 and 9 schematic drawings showing the inside of the glove sanitizer from above, according to some embodiments of the present invention. FIG. 10 is a perspective drawing of a glove sanitizer which includes a disinfectant tank, according to some embodiments of the present invention. FIG. 11 is an isometric view of a cutout of the glove sanitizer, according to some embodiments of the present invention. FIG. 12 is a block diagram showing a control unit controlling different elements of the glove sanitizer according to inputs, according to some embodiments of the present invention.

A glove sanitizer 50 includes a case 102, a glove removal apparatus 200, a ventilation system 300, and a lighting system 400. The case 100 defines an inner cavity 101 and has a front wall 102 with at least one opening 104 configured for being traversed by a user's hand, thereby allowing insertion of a gloved hand 150 (including the hand 152 wearing the glove 154) into the case 100. The case may have two openings 104, to allow insertion of two gloved hands into the case simultaneously.

The glove removal apparatus 200 is configured for removing the glove 154 from the hand 152 and retain the glove 154 inside the case when the hand 152 is moved out of the case 100. The glove removal apparatus 200 includes a curved pipe 202 and a pump 204.

The curved pipe 202 has suction holes 206 positioned along the pipe. Each suction hole 206 is positioned to contact a respective fingertip of the glove 154 when the gloved hand 150 is fully inserted into the case 100. The pump 204 is configured to suck air from the curved pipe 202, thereby lowering pressure in the pipe, causing the suctioning the tips of the glove 154. The pipe may be closed at the end farthest from the pump, to enable suction to occur at the suction holes 206.

The ventilation system 300 is configured to direct an air flow at the glove retained inside the case 100, to dry an outer surface of the glove 154. The ventilation system may include an air pump (not shown) and one or more nozzles 302 in fluid communication with the air pump and configured to direct air toward the glove in the case 100. Alternatively or additionally, the ventilation system 300 may includes or more fans 304 disposed inside the case to circulate air within the case, as seen in FIG. 11. The ventilation system may include one or more heaters 306 disposed inside the case to heat the air inside the case and enhance the drying to the glove, as seen in FIG. 11. Non-limiting examples of the heater(s) 306 includes resistance heaters and Peltier heaters.

The lighting system 400 is configured to emit light inside the case having a predetermined wavelength or combination of wavelengths to illuminate and thereby disinfect the glove 154 inside the case 100. The lighting system 400 includes one or more first lamps 402, located inside the case 100 and configured to illuminate the glove 154 inside the case. The power and wavelength of the light may be chosen to have germicidal properties. For example, the first lamps 402 may emit ultraviolet (UV) light and/or visible light at 415 nm (±10%).

In some embodiments of the present invention, the glove sanitizer 50 also includes at least one tab 500 extending inside the case 100 from the front wall 102, near the at least one opening 104. The tab 500 is configured to enter a space inside a cuff of the glove 154. The tab has an outlet 502 configured to blow air into the glove 154 to separate the glove 154 from the hand 152 and to dry an inside of the glove. The outlet 502 may be in communication with the air pump of the ventilation system or with a respective air pump 503, driving air through the outlet. The tab may further include a second lamp 504 configured to emit light inside the case having a predetermined wavelength or combination of wavelengths to disinfect the inside of the glove.

In some embodiments of the present invention, each tab 500 has an upper surface facing a ceiling 106 of the case and a lower surface facing a floor 108 of the case. The outlet 502 may be located on the top surface of the tab, such that a direction of the air blown by the outlet is at least partially upwards in a direction of the ceiling. The second lamp 504 may be on the upper surface as well.

In some embodiments of the present invention, the case 100 has a transparent ceiling 106, This enables a user to see through the ceiling 106 and move the user's gloved hand 150 inside the case 100 to position the fingertips of the glove 154 to contact the respective suction holes 206.

In some embodiments of the present invention, the glove sanitizer 100 includes a disinfectant tank 800 and a disinfectant delivery system 802. The disinfectant tank 800 is configured to contain a disinfectant liquid. The disinfectant delivery system 802 is configured to direct a spray of the disinfectant liquid onto the glove inside the case. The disinfectant delivery system may include a liquid pump (not shown) and one or more splay nozzles 804. The liquid pump is in fluid communication with the disinfectant tank 800 and with the spay nozzles 804 and is configured to draw the disinfectant liquid from the disinfectant tank 800 and direct the disinfectant liquid to the spray nozzles at a desired pressure, such that the disinfectant liquid is sprayed into the case's cavity.

In some embodiments of the present invention, the disinfectant tank 800 is located in a recess located behind a rear wall 109 upon which the nozzles 302 and he liquid nozzles 804 are disposed. In some embodiments of the present invention, the disinfectant tank 800 has a removable cap 806, which may be removed for filling the disinfectant tank 800 with the disinfectant liquid. In some embodiments of the present invention, the ceiling 106 has a hole through which the disinfectant tank 800 protrudes, as seen in FIG. 10. The disinfectant tank 800 may be removable from the case. In this case, a damaged tank 800 may be replaced, for example.

The glove sanitizer may include one or more sensors 600 for sensing entry of the gloved hand 150 into the case and, responsive to the sensing, activate the pump. The sensor(s) 600 may be located at or near the opening(s) 104. The sensor(s) may include any type of sensors that can detect presence of an object located inside the opening(s). A non-limiting example of such sensor(s) may include infrared (IR) sensor(s) known in the art.

In some embodiments of the present invention, the glove sanitizer 50 includes a control unit 700. The control unit 700 is configured to activate the pump 204 to start suction and the air pump connected to the outlet 502. The activation may be via a user interface 702 (e.g., an on/off button, a plurality of buttons, a keypad, or any other type of user interface), or via detection by the sensor(s) 600 of the user's hand.

Once the user's hand has left the case 100 and the glove 154 is retained, the user may user the interact with the user interface 702 (e.g., push a button) to cause the control unit 700 to activate the lighting system and the lamp of the tab and ventilation system. In this manner, the glove is dried by air blown inside and outside the glove, and is illuminated both inside and outside. If a disinfectant delivery system 802 is present, the user's interaction with the user interface also activates the disinfectant delivery system 802 to spray disinfectant onto the outside of the glove. The sterilization of the glove(s) may be completed after a predetermined time has passed, shutting down the sterilizer automatically at the end of the predetermined time period.

In FIG. 2, the gloved hand 150 is inserted into the case 100 via the opening 104. The sensor 106 (if present) senses the hand traversing the opening 104, and triggers the control unit 700 to activate the pump 204. Alternatively, pump 204 is activated by the control unit 700 via a user input (e.g., interaction with the user interface 702). The control unit may also activate a pump blowing air our of the outlet 502.

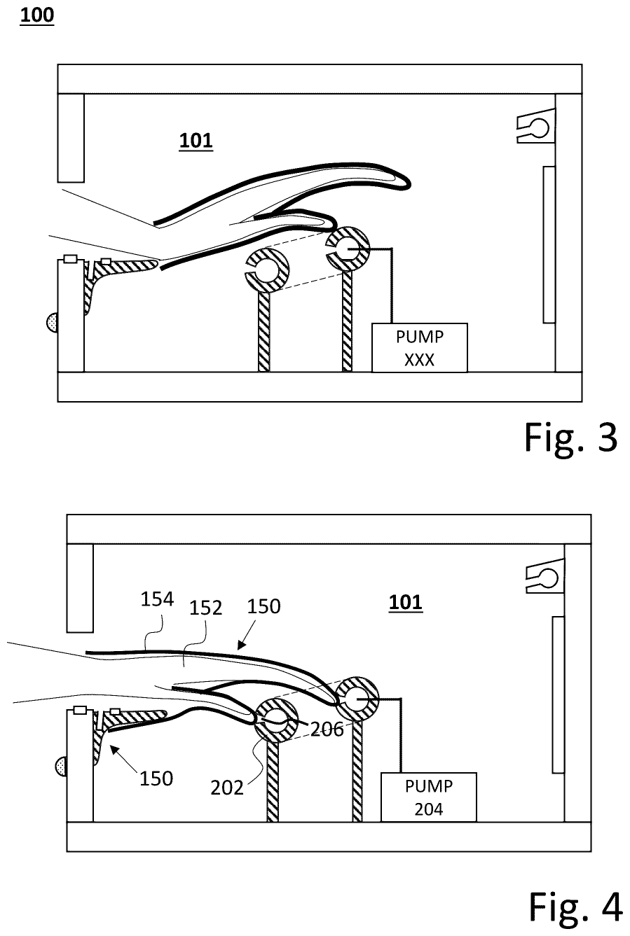

In FIG. 3, the user positions the gloved hand 150 so that the cuff of the glove 154 is proximal to the forward end of the tab 500. In FIG. 4, the user moves the gloved hand 105 backward so the tab 500 is located inside the glove 154, between the glove 154 and the hand 152. The user's fingers are moved so that the glove's fingertips contact respective suction holes 206. The pump 204 suctions air from the pep 202. In this manner, the fingertips of the glove are retained.

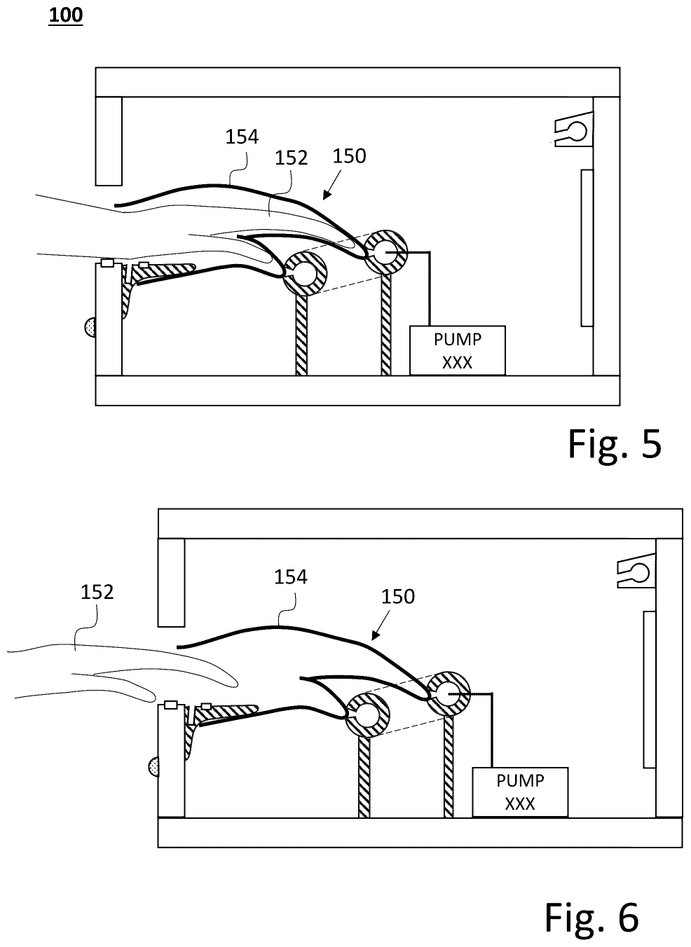

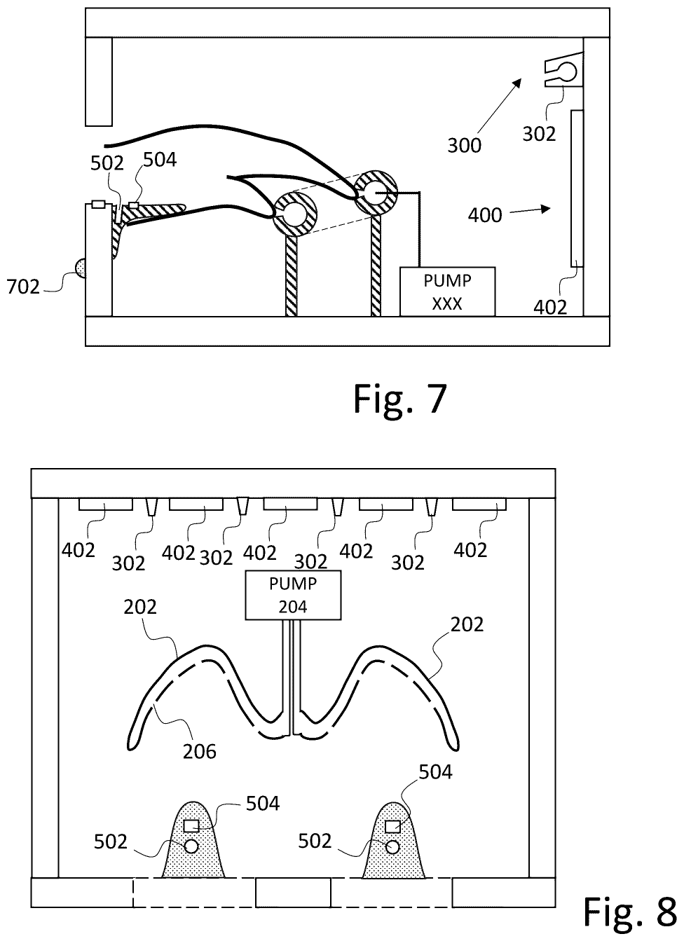

In FIG. 5, air from the outlet 502 blows inside the glove, to separate the glove 154 from the hand 152. This allows the user to retract the hand 152, while the glove 154 is retained inside the case, as seen in FIGS. 6 and 7.

In FIG. 7, when the hand is fully outside the case, the user interface 702 is manipulated to cause the control unit activate the ventilation system, the lighting system, and the second lamp on the tab. In this manner, the glove 154 is dried on the outside of the by air blown through the nozzles 302 and dried on the inside by air blown through the outlet 502. Germicidal light from the first lamp(s) 402 illuminates the outside of the glove 154, while germicidal light from the second lamp(s) 504 illuminates the inside of the glove 154. In this manner the glove is dried and disinfected from inside and outside.

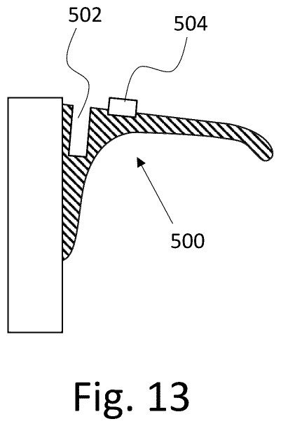

In some embodiments the speed and orientation of the air blown into the glove 154 by the outlet 502 push the bottom of the glove's cuff against the lower surface of the tab 500, thereby increasing friction between the glove's cuff and the tab, such that the glove 154 is maintained in position during drying and disinfection. Additionally or alternatively, the tab 500 may be tilted downward, so that when air blows upward from the outlet 502, the friction between the cuff and the tab is enhanced. The forward end of the tab my be tilted further downward to further enhance the friction between the cuff and the tab when air blows upward from the outlet 502. This can be seen, for example, in FIG. 13.

The glove sterilizer of the present may be a stand-alone device powered by the electrical grid or by a battery. The glove sterilizer of the present invention may be configured to be integrated with a motor vehicle, to be powered by the vehicle's battery.

What is claimed is:

1. A glove sanitizer, comprising:
i) a case having a wall with at least one opening configured for insertion of a gloved hand therethrough;
ii) a glove removal apparatus, for removing the glove from the gloved hand and retain the glove inside the case when the hand is moved out of the case, wherein the glove removal apparatus comprises:
a curved pipe having suction holes positioned along the pipe, each suction hole being positioned to contact a respective fingertip of the glove when the gloved hand is fully inserted into the case;
a pump configured to suck air from the curved pipe, thereby suctioning the tips of the glove at the suction holes;
iii) a ventilation system, configured to direct an air flow at the glove retained inside the case, to dry an outer surface of the glove;
iv) a lighting system, configured to emit light inside the case having a predetermined wavelength or combination of wavelengths to disinfect an outside of the glove inside the case.

2. The glove sanitizer of claim 1, further comprising:
v) at least one tab extending inside the case near the at least one opening, the tab being configured to enter a space inside a cuff of the glove and having an outlet configured to blow air into the glove to separate the glove from the hand and to dry an inside of the glove.

3. The glove sanitizer of claim 2, wherein the tab further comprises a lamp configured to emit light inside the case having a predetermined wavelength or combination of wavelengths to disinfect the inside of the glove.

4. The glove sanitizer of claim 2, wherein the at least one tab has an upper surface facing a ceiling of the case and a lower surface facing a floor of the case, and wherein:
the outlet is located on the upper surface of the tab, such that a direction of the air blown by the outlet is at least partially upwards in a direction of the ceiling.

5. The glove sanitizer of claim 1, further comprising:
a disinfectant tank configured to containing a disinfectant liquid; and
a disinfectant delivery system configured to direct a spray of the disinfectant liquid onto the glove inside the case.

6. The glove sanitizer of claim 1, wherein the case has a transparent ceiling, to enable a user to see through the ceiling and move the user's gloved hand inside the case to position the fingertips of the glove to contact the respective suction holes.

7. The glove sanitizer of claim 1, further comprising a sensor for sensing entry of the gloved hand into the case and, responsive to the sensing, activate the pump.

8. The glove sanitizer of claim 7, comprising a control unit configured to:
responsive to a sensing of an entry of the gloved hand by the sensor, activate the pump and the outlet assembly to remove the glove from the user's hand;
responsive to a user input, disinfect and dry the glove retained inside the case, by:
activating the lighting system;
activating the ventilation system.

9. The glove sanitizer of claim 8, further comprising:
a disinfectant tank configured to containing a disinfectant liquid; and
a disinfectant delivery system configured to direct a spray of the disinfectant liquid onto the glove inside the case;
wherein, responsive to a sensing of an entry of the gloved hand by the sensor, the control unit is configured to activate the pump and the outlet assembly to remove the glove from the user's hand;
responsive to a user input, the control unit is configured to disinfect and dry the glove retained inside the case, by:
activating the disinfectant delivery system;
activating the lighting system;
activating the ventilation system.

10. The glove sanitizer of claim 7, further comprising:
at least one tab extending inside the case near the at least one opening, the tab being configured to enter a space inside a cuff of the glove and having an outlet configured to blow air into the glove to separate the glove from the hand and to dry an inside of the glove, the tab further comprises a lamp configured to emit light inside the case having a predetermined wavelength or combination of wavelengths to disinfect the inside of the glove;
wherein, responsive to the user input, the control unit is configured to disinfect and dry the glove retained inside the case, by:
activating the lighting system and the lamp of the tab;
activating the ventilation system.

11. The glove sanitizer of claim 10, further comprising:
a disinfectant tank configured to containing a disinfectant liquid; and
a disinfectant delivery system configured to direct a spray of the disinfectant liquid onto the glove inside the case;
wherein, responsive to a sensing of an entry of the gloved hand by the sensor, the control unit is configured to activate the pump and the outlet assembly to remove the glove from the user's hand;
responsive to a user input, the control unit is configured to disinfect and dry the glove retained inside the case, by:
activating the disinfectant delivery system;
activating the lighting system and the lamp of the tab;
activating the ventilation system.
